(19) United States
(12) Patent Application Publication
(10) Pub. No.: US 2003/0077265 A1
(43) Pub. Date: Apr. 24, 2003

(54) Isolation and purification of proteinase inhibitor ll

(76) Inventors: Rod Ausich, Des Moines, IA (US); Robert Stomp, Des Moines, IA (US); Fayad Z. Sheabar, West Des Moines, IA (US)

Correspondence Address: Daniel A. Rosenberg, The Financial Center, Suite 2500, 666 Walnut Street, Des Moins, IA, 50309, US

(21) Appl. No.: 09/900,058

(22) Filed: Sep. 13, 2001

(51) Int. Cl. A61K 38/43; C12N 9/99; A61K 35/78

(52) U.S. Cl. 424/94.1; 435/184; 424/725

(57) ABSTRACT

The present invention provides an ultrafiltration method for isolation and purification of a proteinase inhibitor extract from plant tissue, preferably potato tubers. The extraction and isolation of the proteinase inhibitor from potatoes begins with the addition of an organic acid, preferably formic acid, and a salt, preferably sodium chloride, to raw potatoes. The mixture is subjected to process steps to extract soluble proteins. Undesired proteins are denatured and removed, resulting in an extract solution suitable for ultrafiltration. The ultrafiltration process comprises two parts, concentration and diafiltration. The concentration part is primarily for dewatering, and as such, acids, salts, and other small molecules are removed concurrent to concentration. An ideal ultrafiltration membrane composition comprises regenerated cellulose with a nominal molecular weight cut-off of 10,000 Dalton. A concentration factor of 10 times volume of the clarified extract volume is achieved using a 100 mM ammonium bicarbonate solution.

### BACKGROUND OF THE INVENTION

1. Field of the Invention

The present invention relates to the isolation and purification of a proteinase inhibitor, and more specifically, to the isolation and purification from whole potatoes of Proteinase Inhibitor-II (PI2) by ultrafiltration with ammonium bicarbonate solutions.

2. Background of the Prior Art

The isolation and purification of plant-derived proteins is well known in the field of biochemistry. In 1972, Melville and Ryan reported a large-scale preparation for isolating Chymotrypsin Inhibitor I from potato tubers (Melville, J. C. and Ryan, C. A. Chymotrypsin inhibitor I from potatoes. J. Biological Chem., 247: 3445-3453, 1972). According to the method of Melville and Ryan, potatoes were sliced with peels intact and soaked in a sodium dithionite solution, homogenized, and expressed through nylon cloth. The resulting juice was adjusted to a pH of 3, centrifuged at 1000×g for 15 minutes at 5° F., and the supernatant collected and fractionated with ammonium sulfate.

Purification was achieved through water washing and heat treatment whereby clear filtrates of heated fractions were pooled and lyophilized. Suspending the lyophilized powder in water, dialyzing it against water for 48 hours, and lyophilizing the resulting clear filtrate obtained a crude extract. Resuspended extract was then centrifuged and applied to a column of Sephadex G-75. Collected fractions containing the Inhibitor I were pooled, evaporated, and desalted on a column of Sephadex G-25. The resulting gel-filtered inhibitor product was determined to be approximately 90% Inhibitor I protein purified by dissociation on a Sephadex G-75 column and desalted on a column of Sephadex G-25.

The Ryan lab followed-up by reporting the isolation and characterization of Proteinase Inhibitor II in much the same manner as described for Inhibitor I (Bryant, J., Green, T. R., Gurusaddaiah, T., Ryan, C. L. Proteinase inhibitor II from potatoes: Isolation and characterization of its protomer components. Biochemistry 15: 3418-3424, 1976). Bryant et al. differentiated potato-derived proteinase inhibitors into two groups based on their respective stabilities to a temperature of 80° C. for 10 minutes. Proteinase Inhibitor I (PI₁) is characterized as a tetrameric protein composed of four hybridized isoinhibitor protomer species having a molecular weight of 39,000, whereas PI2 is characterized as a dimeric inhibitor comprising four isoinhibitor promoter species having a molecular weight of 21,000.

The isolation of proteinase inhibitor proteins from potatoes is described in WO 99/01474. Proteins from potato tubers are extracted in soluble form in an aqueous/alcohol extraction medium, such as dilute formic acid and 20% ethanol. The alcohol extract is heated to a first temperature to denature most of the unwanted proteins and cooled to a second temperature to form a precipitate phase constituting the debris and a soluble phase that contains the heat stable proteinase inhibitor proteins. The heat stable proteinase inhibitor proteins are precipitated from the soluble phase by dialysis against a suitable dialysis medium, such as dilute formic acid.

Recently, PI2 has been implicated in playing a role in extending satiety in subjects fed a nutritional drink composition containing PI2. U.S. patent application Ser. No. 09/624,922 describes that subjects reported a significant reduction in hunger for up to 3½ hours post meal when fed a meal comprising a nutritional drink composition containing PI2. Likewise, fullness ratings were enhanced, and each study subject lost an average of 2 kg over a 30-day period without experiencing the adverse side effects typically associated with appetite suppressing compounds. Mechanistically, it is thought that as a trypsin and chymotrypsin inhibitor, when consumed by a subject, PI2 stimulates the release of endogenous cholecystokinin, a known putative feedback agent effective in reducing the desire to intake food. Accordingly, a need exists for a large-scale isolation and purification process to extract PI2 in a cost-effective and efficient manner meeting industrial qualitative and quantitative standards.

A technique capable of large-scale isolation and purification is ultrafiltration, a type of membrane filtration and separation technique that utilizes membranes having pore sizes between 0.001 and 0.1 μm. Methodologies utilizing ultrafiltration are particularly useful for concentrating dissolved molecules such as proteins, peptides, nucleic acids, carbohydrates, and other biomolecules, as well as desalting, exchanging buffer, and gross fractionation. Diafiltration is a selective fractionation process of washing smaller molecules through a membrane, while leaving the larger molecule of interest in the retained solution, also known as retentate. In selecting a membrane suitable for filtering a target molecule, the molecular weight cutoff (MWCO) of a membrane is utilized to define the ability of the membrane to exclude molecules on a size basis. 90% of an ideally globular molecule. MWCO is the size designation (in kilodaltons “KD”) for ultrafiltration membranes. The term Nominal Molecular Weight Cutoff (NMWCO) is defined as a membrane's ability to retain 90% of an ideally globular molecule having the designated molecular weight.

As discussed above, ultrafiltration is a technique used for the separation under elevated pressure of dissolved molecules in solution on the basis of size. Molecules larger than the pore size of the membrane will not pass through the membrane surface and will remain in the retentate, and may further be retained on the surface of the membrane. Accumulations of retained molecules on the membrane typically form a gel layer, significantly reducing the separation performance characteristics of the membrane. Separation capacity of a given ultrafiltration system is a function of the ability of the selected membrane to allow the smaller particles to pass through the membrane in the permeate, while minimizing gel formation in the retentate. However, a direct correlation between a molecular weight and size does not always exist.

Molecular conformations (including both intermolecular and intramolecular interactions) can significantly alter the apparent size of a molecule. Having numerous modes of interaction available, proteins are particularly susceptible to conformational changes while dissolved in solution. Solution characteristics such as pH, solute concentration, temperature, and ionic strength significantly affect the apparent size of particles and molecules in solution, and therefore affect conformational characteristics as well.

### SUMMARY OF THE INVENTION

Ultrafiltration processing required for the isolation of PI2 creates complex and dynamic solution compositions affecting membrane capacity and performance. Ultrafiltration of a PI2 extract comprises two parts, concentration and diafiltration. The concentration part is primarily for dewatering, and as such, acids, salts, and other small molecules are removed concurrent to concentration. Through the course of ultrafiltration, each of the solution constituents will have different separation dynamics, and the overall filtrate mixture profile will change as the acid and salt concentrations change. Similarly, the chemistry of the retained molecules changes as retained molecular concentration increases. As the extractant mixture is concentrated, permeate flux rates decline.

The permeate flux rate is a function of both gel formation on the membrane and the retentate mixture profile. Diafiltration is provided to increase the available liquid volume which in turn increases the amount of permeate generated, thereby allowing for a more thorough removal of impurities passing through the membrane. Addition of an ammonium bicarbonate solution to the concentrated mixture in the ultrafiltration method of the present invention has been found to be more effective in improving membrane performance than previously anticipated. Ammonium bicarbonate was originally chosen as a diafiltration buffer to exploit its capacity as a neutralizer of the residual acidic components of the concentrated solution. Ammonium bicarbonate was also selected to bind the target protein, thereby displacing the previously bound and undesirable sodium chloride from the target protein. Furthermore, ammonium bicarbonate is a preferred buffer due to the ease of which it is removed during lyophilization.

While the anticipated benefits of an ammonium bicarbonate buffer system were ultimately recognized under experimental conditions, utilization of the buffer system produced unexpected benefits. For example, the permeate flux rate increased during the diafiltration process, an unexpected benefit resulting in an overall increase to the capacity of the ultrafiltration process. Increased capacity allows for removal of additional impurities, thereby increasing final product purity. While it is not completely understood, the benefits attributable to the ammonium bicarbonate diafiltration buffer are thought to be a function of the ionic environment generated when ammonium bicarbonate is present. The observed benefits of using ammonium bicarbonate as the diafiltration buffer are not reported elsewhere and were not predicted prior to experimentation.

### BRIEF DESCRIPTION OF THE DRAWINGS

FIG. 1 is a process flow diagram outlining process steps and sampling points identified in the isolation of PI2 from whole potatoes, as described in Example 1 below.

### DETAILED DESCRIPTION OF THE INVENTION

The extraction and isolation of PI2 from potatoes begins with the addition of an organic acid, preferably formic acid, and a salt, preferably sodium chloride, to raw potatoes. The mixture is subjected to comminution to reduce the particle size of the potato particles and extract soluble proteins. Centrifugation is used to remove solids and the liquid fraction is heated at a temperature sufficient to denature many undesired proteins but not PI2. The solution is again centrifuiged to remove the insoluble denatured proteins and the liquid fraction is microfiltered to remove relatively large particles. Ultrafiltration is used to further purify the PI2 in the retentate.

Ultrafiltration of the clarified extract from the PI2 isolation process is the most crucial step in the isolation and purification procedure as multiple goals are accomplished using a comprehensive separation method. Clarified PI2 extract is concentrated, or dewatered, through ultrafiltration. Ultrafiltration also serves to remove low molecular weight impurities, including sugars and proteins. Sodium chloride and formic acid used in the extraction steps are also removed during ultrafiltration. To accomplish the goals of concentration and impurity removal while maintaining cost-effectiveness and full production capability, a thorough examination of a multitude of factors directed to the ultrafilter design, composition, and process condition was performed. The acidic nature of the clarified liquid requires an ultrafiltration membrane composed of chemically rigorous materials. The size of the target protein dictates the NMWCO of the membrane system. Maximizing the concentration factor is sought, while maintaining membrane lifetime and integrity. Membrane flow rates are critical, as increases in concentration directly correlates to decreases in flow rates. Subsequently, decreases in flow rates lead to increases in the costs associated with membrane and ultrafilter equipment, as well as lengthens process times. To remove the acid content of the clarified extract, an appropriate buffer system is required. The chemical nature of the buffer must not adversely affect final product composition, while its concentration needs to balance pH change capacity, and membrane compatibility. As low molecular weight impurities are removed through the entire ultrafiltration process, the volume of buffer used in the product dialysis was minimized for cost effectiveness, while achieving desired overall final protein purity.

### EXAMPLE 1

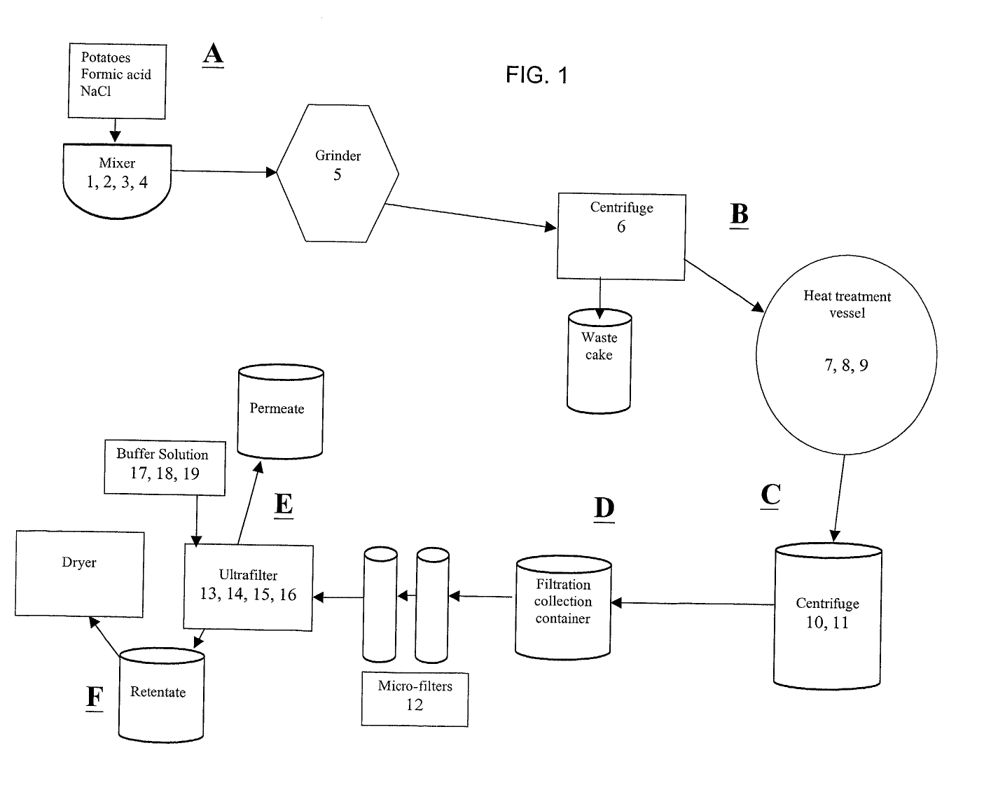

A process for the isolation of PI2 from whole potatoes was developed in an attempt to maximize yield, minimize impurities, minimize cost, and achieve commercial feasibility. Ultrafiltration of clarified PI2 extract was examined by monitoring the following variables: membrane composition, nominal molecular weight cut-off, concentration factor, flow rate, diafiltration buffer choice, buffer concentration, and diafiltration solution volume. In a preferred embodiment, the membrane composition is regenerated cellulose, the nominal molecular weight cut-off is 10,000 Dalton, the concentration factor achieved is 10 times the original volume, the flow rate is 45 liters/(meter²-hour) (LMH) at a 20 psig retentate pressure using an ammonium bicarbonate (NH₄HCO₃) buffer system, the diafiltration buffer is 100 mM, and 6 times the concentrated volume is used as diafiltration feed. The variables of the preferred embodiment correlate to process steps 13, 14, 15, 16, 17, 18, 19, respectively, from the manufacturing process flow diagram (FIG. 1).

Methods/Materials

As stated above, the current process for the extraction and purification of PI2 from whole, raw potatoes includes at least nineteen separate process steps. FIG. 1 represents a diagram of the process of the present invention. The diagram is labeled by identifying the nineteen identified steps (FIG. 1, steps 1-19). The diagram also identifies six sampling points for monitoring extraction efficiency (FIG. 1, Points A-F). The nineteen process steps are as follows (FIG. 1, Factors 1-19): (1) raw material choice; (2) formic acid content of extractant; (3) sodium chloride content of extractant; (4) ratio of extractant to raw potato employed; (5) grind profile of the final slurry; (6) screen mesh of the bulk centrifuge; (7) maximum temperature of the material during heat treatment; (8) hold time of heat treatment step; (9) final temperature of cooling stage; (10) hold-time/force applied in centrifuge clarification; (11) percent solids allowed in clarified extract; (12) pore size of microfilter; (13) membrane composition; (14) molecular weight cut-off; (15) concentration factor; (16) flow rate; (17) diafiltration buffer choice; (18) buffer concentration; (19) volume diafiltered against. The six monitoring points are as follows (FIG. 1, Points A-F): (A) whole, raw potatoes; (B) centrifuge filtrate; (C) post-heat treat; (D) clarified extract; (E) permeate; (F) diafiltered extract.

Process Steps (FIG. 1, Steps 1-19)

1. Raw Material Choice

Whole, raw potatoes are used for the protease inhibitor feedstock. Russet Burbank, Russet Norkotah, and Norkotah varieties of potatoes were utilized to determine which variety resulted in the highest yields. Each variety was examined with respect to consistently producing through extraction the highest quantities of protease inhibitors, including PI2.

2. Formic acid content

Formic acid is a useful and appropriate component of the extractant formula. Many organic acids were examined for extraction efficiency and formic acid consistently exhibited the greatest cost-effectiveness and its concentration in the extractant solution was optimized through experimentation.

3. Sodium chloride content

To increase the solubility of the potato proteins, sodium chloride is a useful and appropriate component of the extractant formula. Its concentration in the extractant solution was optimized through experimentation.

4. Extractant to raw potato ratio

The process does not recycle any of the extractant used to isolate PI2, or separated fractions containing undesirable impurities. The amount of extractant used was minimized as additional extractant represents additional material cost, increased processing time, and increased energy consumption.

5. Grind profile of the final slurry

The grind profile of the final slurry has significant impact on the yield of PI2 realized from the isolation process. The grind profile is controlled by the gap-width of the rotary grinding head and total grind time (Comitrol Processor, Urschel Laboratories, Valparasio, Ind.).

6. Screen mesh of the bulk centrifuge

The final slurry is separated into two fractions using a BIRD Tolhurst cage-type filter centrifuge (Baker Hughes, Incorporated, Huston, Tex.). This device allows for the collection of a liquid filtrate that contains the PI2 and other soluble proteins and impurities while removing the bulk fiber and starch found in potatoes.

7. Maximum temperature of the material during the heat treatment

The filtrate from the Tolhurst centrifuge is heat treated to alter the solubility of some protein impurities that are present.

8. Hold time of the heat treatment

The heat treatment of the filtrate is performed over a period of time sufficient to allow for separation of the non-heat stable impurities.

9. Final temperature of the cooling stage

The solubility of the impurities is also diminished by correspondingly decreasing the temperature of the heat-treated filtrate. Significant reduction in solubility is gained by cooling the heat-treated liquid to below 26° C.

10. Hold-time/force applied in centrifuge clarification

The heat-treated filtrate contains once soluble impurities in dispersion. A high gravity clarifier can remove these impurities from the supenatant. A Sharples AS-16 tubular bowl clarifier is used to remove the impurities from the heat-treated filtrate (Sharples Tubular Centrifuge, Model AS-16, Universal Process Equipment, Inc., Robbinsville, N.J.).

11. Percent solids allowed in clarified extract

The feed rate of material into the clarifier affects the final profile of the clarified extract. A greater feed rate allows for more rapid processing, but allows a correspondingly greater percentage of solids to remain in the clarified extract. As the percentage of solid content rises in the clarified extract, the particle size of the suspended solids increases proportionately. Both the size and amount of suspended solids affects the efficiency of the microfilter.

12. Pore size of microfilter

The target protein (PI2) is smaller than 0.1 micron when in solution. All particles in the extract larger than 0.1 micron are impurities, and require removal. Having the potential to foul the membrane of the ultrafilter, the clarified extract is microfiltered to remove particles that are not removed by the clarifier.

13. Membrane composition

Ultrafiltration membranes of varying composition were explored for chemical compatibility, separation efficiency, and rigor.

14. Molecular weight cut-off

The molecular weight of PI2 is approximately twenty-one kilo Dalton (KD). In addition to PI2, low molecular weight impurities remain in the extract. The ultrafilter serves to remove many types of undesirable compounds from the extract.

15. Concentration factor

Reducing the total volume of the extract directly reduces the cost of the final drying of the crude PI2 extract. The concentration stage of the ultrafiltration serves to remove acid, salt, water, and other low-molecular weight molecules. The limiting factors are the solubility of the large molecular weight compounds (including PI2) and the flow rate across the membrane.

16. Flow rate

The cross membrane flow rate (of retentate through the ultrafilter) was optimized to maximize the permeate collection rate.

17. Choice of buffer for diafiltration

A diafiltration buffer is selected by considering its qualities with respect to pH, its ability to exchange salts, and the ease by which it is removed from the desired end product.

18. Buffer concentration

Use of the diafiltration buffer was minimized due to cost and disposal considerations.

19. Volume of buffer used in diafiltration

In conjunction with the strength of the buffer used, the number of washes (relative volumes diafiltered against) was studied.

Results

Membrane Selection

An experiment was conducted to determine if the ultrafiltration membranes were subject to fouling. The retentate flow and back pressure were held constant and the flux measured over time. The system was in total reflux mode and the initial flux reading was made after running the system for 5 minutes to allow for equilibration. If the flux remained constant with time, no fouling was occurring and the back pressure was then increased. Three types filters tested, the Pall Filtron Centramate CS010C12, (Pall Corporation, East Hills, N.Y.), the Pall Filtron Maximate CS010G02, (Pall Corporation, East Hills, N.Y.), and the A/G Technology UFP-5-C-4A, (A/G Technology Corporation, Needham, Mass.). The Maximate cartridge uses the same membrane as the Centramate so protein retention was expected to be identical. However the Maximate cartridge is supplied in a longer path length format that is less expensive to produce and allows for the production of a single pump ultrafiltration unit suitable for our process. Using the Centramate would require multiple pumps and much higher initial capital costs.

Using the Pall Filtron Centramate filter, retentate flow was set at 825 ml/min, while inlet pressure was varied from 27 psig to 42 psig. Outlet pressure varied from 10 psig to 31 psig, and the flux varied from 79 to 124 ml/min. No strong evidence of fouling at higher backpressures was observed. At 42 psig, the pump had reached its limit and higher pressures were not examined. For purposes of further testing, it was determined that an outlet pressure of 20 psig was a good compromise for the back pressure as there was only a 6% difference in flux at higher pressures. The reported differences do not provide significant variation in the cost or specifications of the ultrafiltration equipment.

Using the A/G Technologies filter, retentate flow was set at 1200 ml/min, while inlet pressure was varied from 12 psig to 43 psig. Outlet pressure varied from 11 psig to 40 psig, and the flux varied from 13 to 30 ml/min. No evidence of fouling at higher backpressures was observed. Once again, the pump reached its limit at an outlet pressure of 40 psig, so higher pressures were not examined. For purposes of further testing, it was decided to accept 40 psig as the experimental back pressure as the flux remained quite low in comparison to an observed 20% increase in flux at the higher back pressures.

Using the Pall Filtron Maxisette filter, retentate flow was set at 800 ml/min, while inlet pressure was varied from 24 psig to 40 psig. Outlet pressure varied from 10 psig to 30 psig, and the flux varied from 190 to 314 ml/min. No evidence of fouling at higher backpressures was observed. Once again, the pump reached its limit at an outlet pressure of 40 psig, so higher pressures were not examined. For purposes of further testing, it was determined that an outlet pressure of 20 psig was a good compromise for the back pressure as here was less than a five percent difference in flux at higher pressures.

In the next stage of testing, each ultrafilter system was used 3 times to concentrate and diafilter 4 liters of extract, further testing fouling and performance characteristics. One hundred mM ammonium bicarbonate was used as the diafiltration buffer.

TABLE 1
Composite of Concentration Phase with
Pall Filtron Centramate CS010C12

| Retentate flow | Inlet Pressure | Outlet Pressure | Flux | Volume |
|---|---|---|---|---|
| (ml/min) | (psig) | (psig) | (ml/min) | (ml) |
| 800 | 34 | 20 | 119 | 4000 |
| 800 | 35 | 20 | 107 | 2000 |
| 800 | 36 | 20 | 91 | 1000 |
| 800 | 38 | 20 | 65 | 400 |

TABLE 2
Composite Diafiltration Phase with Pall
Filtron Centramate CS010C12

| Retentate flow | Inlet Pressure | Outlet Pressure | Flux | Buffer |
|---|---|---|---|---|
| (ml/min) | (psig) | (psig) | (ml/min) | volumes |
| 783 | 39 | 20 | 53 | 1 |
| 783 | 39 | 20 | 57 | 2 |
| 800 | 40 | 20 | 56 | 3 |
| 792 | 39 | 20 | 57 | 4 |
| 800 | 39 | 20 | 58 | 5 |
| 783 | 39 | 20 | 60 | 6 |

TABLE 3
Composite of Concentration Phase with
A/G Technologies UFP-5-C-4A

| Retentate flow | Inlet Pressure | Outlet Pressure | Flux | Volume |
|---|---|---|---|---|
| (ml/min) | (psig) | (psig) | (ml/min) | (ml) |
| 1200 | 41 | 40 | 29 | 4000 |
| 1200 | 43 | 40 | 23 | 2000 |
| 1233 | 45 | 41 | 22 | 1000 |
| 1317 | 48 | 40 | 20 | 400 |

TABLE 4
Composite of Diafiltration Phase with
A/G Technologies UFP-5-C-4A

| Retentate flow | Inlet Pressure | Outlet Pressure | Flux | Buffer |
|---|---|---|---|---|
| (ml/min) | (psig) | (psig) | (ml/min) | volumes |
| 1258 | 48 | 40 | 19 | 1 |
| 1308 | 18 | 40 | 17 | 2 |
| 1325 | 48 | 40 | 18 | 3 |
| 1333 | 49 | 40 | 19 | 4 |
| 1333 | 49 | 41 | 20 | 5 |
| 1333 | 48 | 40 | 20 | 6 |

TABLE 5
Composite Concentration Phase with Pall
Filtron Maxisette CS010G01

| Retentate flow | Inlet Pressure | Outlet Pressure | Flux | Volume |
|---|---|---|---|---|
| (ml/min) | (psig) | (psig) | (ml/min) | (ml) |
| 800 | 32 | 20 | 250 | 4000 |
| 800 | 32 | 20 | 231 | 2000 |
| 813 | 33 | 20 | 204 | 1000 |
| 838 | 40 | 21 | 169 | 400 |

TABLE 6
Composite Diafiltration Phase with Pall
Filtron Maxisette CS010G01

| Retentate flow | Inlet Pressure | Outlet Pressure | Flux | Buffer |
|---|---|---|---|---|
| (ml/min) | (psig) | (psig) | (ml/min) | volumes |
| 800 | 35 | 21 | 132 | 1 |
| 800 | 35 | 20 | 150 | 2 |
| 800 | 35 | 20 | 150 | 3 |
| 800 | 34 | 20 | 151 | 4 |
| 800 | 34 | 20 | 149 | 5 |
| 800 | 34 | 21 | 151 | 6 |

Based upon the composite profiles represented in Tables 1-6, it can be calculated that the average flux for the AIG Technology membrane is 20.2 liters/meter²-hour (LMH). The average flux for the Pall Filtron membrane is 54.70 LMH. The average flux for processes using the Maxisette CS010G01 was 63 LMH.

The following conclusions are drawn from the above experiments:

A) Both Pall Filtron and A/G Technology membranes are non-fouling under tested conditions.

B) The reduced flux of the A/G Technology membrane, 20 LMH, versus the Pall Filtron approximately 60 LMH, necessitates a much larger, and therefore more expensive system if the A/G Technology membrane is used.

C) The increased flow rate achieved using the Maxisette technology is preferable versus the Centramate holder.

Protease Inhibitor Recovery

Final diafiltered solutions from an A/G Technology trial and from a Pall Filtron trial were freeze dried (lyophilized) and the product analyzed for PI2 content. Protein content is determined according to the Bradford protein assay (Bradford, M. M. A rapid and sensitive method for the quantitation of microgram quantities of protein utilizing the principle of protein-dye binding. Anal. Biochem. 72: 248-254, 1976) using the Pierce Coomassie Plus Protein Assay 23236. Five hundred ml of extract was supplied for diafiltration. PI2/kg potato calculation assumed that 100 kg of potatoes yielded 90 kg of extract. A 90 kg extract yield was a standard value for extractions utilizing whole potatoes.

TABLE 7
Relative Protease Recovery

| | | | | PI-2/kg |
|---|---|---|---|---|
| | Gross crude | | Total PI-2 | potato |
| Filter | product (mg) | % PI-2 | (mg) | (mg) |
| A/G Tech UFP-5-C-4A | 630 | 7.60 | 47.88 | 86.2 |
| Pall Filtron Centramate | 800 | 9.61 | 76.88 | 138.4 |
| CS010C12 | | | | |

The A/G Technology membrane retained only 62.3% of the protease inhibitor in comparison to that recovered by the Pall Filtron membrane. Analyses were completed in duplicate and the results reported as an average. A comparison of the respective membranes resulted in a somewhat surprising conclusion. While the A/G Technology membrane is rated at 5,000 NMWCO, and the Pall Filtron membrane is rated at 10,000 NMWCO, there are no industry standards regarding the testing for nominal molecular weight cutoff. Due to differences in the respective pore geometries of the Pall Filtron cellulosic membrane and the A/G Technology polysulfone membrane, passage of protein is not only molecular mass dependent, but also molecular geometry dependent. Further collaborating the relative permeability of the A/G Technology membrane to potato protease inhibitor is the observation that a 10,000 NMWCO filter passes greater than 95% of the protease through the membrane—thus all the tests described above were done using the 5000 NMWCO membrane from A/G Technology. Moving to a 1000 NMWCO membrane resulted in dramatically reduced flux.

Diafiltration Buffer

Diafiltration against water resulted in rapid and irreversible fouling of the membrane with a corresponding flux rate decay. Accordingly, it was necessary to identify a buffer system that would prevent or reduce fouling and be compatible with the concentration step, the composition of the final product, the selected membrane, and the lyophilization step.

Using the conditions determined with the small-scale laboratory ultrafiltration unit the scalability of the process and suitability of our equipment in the pilot scale set up was analyzed. Appropriate process scalability was found with approximately equal conditions of flux and permeate flow rates.

TABLE 8
Initial Water Flux Determinations

| Inlet | Out | Perm. | Ret. | Perm. | Inlet | |
|---|---|---|---|---|---|---|
| pres. | Pres. | Pres. | Flow | Flow | Flow | Flux |
| (psig) | (psig) | (psig) | (ml/m) | (ml/m) | (ml/m) | (ml/m/m²) |
| 6.3 | .1 | 0 | 320 | 12 | 332 | 0.12 |
| 6.3 | 2.1 | 0 | 316 | 30 | 346 | 0.30 |
| 7.6 | 3.7 | 0 | 300 | 50 | 350 | 0.50 |
| 10.1 | 6.6 | 0 | 261 | 75 | 336 | 0.75 |
| 12.9 | 9.7 | 0 | 222 | 115 | 337 | 1.15 |
| 16.5 | 13.6 | 0 | 171 | 200 | 371 | 2.00 |
| 21.9 | 19.4 | 0 | 92 | 245 | 337 | 2.45 |
| 25.8 | 23.7 | 0 | 30 | 210 | 240 | 2.10 |
| 11.9 | 5.7 | 0 | 428 | 103 | 531 | 1.03 |
| 19.2 | 12.9 | 0 | 338 | 200 | 538 | 2.00 |

TABLE 9
Diafiltration of Concentrate Against Buffer

| Inlet | Out | Perm. | Ret. | Perm. | Conduc- | Diafil- | |
|---|---|---|---|---|---|---|---|
| pres. | Pres. | Pres. | Flow | Flow | tivity | tration | Time |
| (psig) | (psig) | (psig) | (ml/m) | (ml/m) | (mS/mol) | volumes | (min) |
| 44.5 | 33.2 | 0 | 524 | 137 | 59.6 | 0 | 0 |
| 44.5 | 34.6 | 0 | 461 | 130 | 26.3 | 1 | 16 |
| 41.5 | 31.0 | 0 | 545 | 123 | 14.4 | 2 | 30 |
| 43.5 | 33.0 | 0 | 557 | 124 | 9.1 | 3 | 47 |
| 43.9 | 33.8 | 0 | 538 | 131 | 7.0 | 4 | 64 |
| 42.2 | 32.4 | 0 | 547 | 125 | 6.0 | 5 | 72 |

The 100 mM ammonium bicarbonate buffer had a conductivity of 5.22 mS/mol. A 6X volume wash appears to be sufficient for removal of salt and increasing product purity.

To prevent fouling and to maintain the membrane, cleaning was performed by flushing with 0.1 N NaOH for 30 minutes with a crossflow of ˜600 ml/minute at 7.7 TMP. Membranes cleaned in accordance with this procedure recovered nearly 100% (99.4%) of initial performance characteristics. Typically, a filter loses 15-20% of its initial performance characteristics after a first use, and thereafter attains, with proper maintenance, a performance plateau that provides consistent and repeatable results for an extended period of use.

TABLE 10
Diafiltration of Concentrate Against Water

| Inlet | Out | Perm. | Ret. | Perm. | Conduc- | Diafil |
|---|---|---|---|---|---|---|
| pres. | Pres. | Pres. | Flow | Flow | tivity | tration |
| (psig) | (psig) | (psig) | (ml/m) | (ml/m) | (mS/mol) | volumes |
| 42.9 | 31.9 | 0 | 512 | 150 | 27.9 | 1 |
| 43.8 | 33.2 | 0 | 500 | 112 | 11.3 | 2 |
| ˜45 | ˜34 | 0 | 500 | 107 | Not taken* | 3 |

*Due to increased pressure and rapidly decreasing permeate flow the trial was halted.

Cleaning with 100 mM NaOH and testing with water revealed a 33% loss of permeate flow, resulting in a pressure drop across the membrane of 8.5 psig, versus 6 psig observed following use against the preferred buffer solution. Diafiltration against a buffer is necessary to prevent blinding of the filter membrane.

The use of 100 mM ammonium bicarbonate during the diafiltration phase of PI2 purification prevents fouling of the cellulosic membrane and allows for removal of a major impurity, carboxypeptidase inhibitor, which elute after PI2 in HPLC. When water is used as a diafiltration buffer, irreversible fouling occurs quickly and the impurity is retained.

Integration values demonstrate that the impurity is contained at about one-third of the concentration of PI2. After concentration of the sample on the ultrafilter, during which time some of the low molecular weight impurities co-eluting with PI2 are removed, the ratio of impurity to PI2 is essentially unchanged. Diafiltration against 6 volumes of 100 mM ammonium bicarbonate results in a substantially reduced ratio of impurity to PI2. Diafiltration against larger volumes of ammonium bicarbonate exhibited almost complete removal of the impurity after 20 volumes. The ratio of impurity to PI2 observed when using water as the diafiltration buffer remained unchanged.

TABLE 11
Effect of Diafiltration with 100 mM Ammonium Bicarbonate

| | P12 Integrated | Doublet Impurity | |
|---|---|---|---|
| | Area | Integrated | Ration Impurity: |
| Sample | (mAU) | Area (mAU) | P12 |
| Heat Treated | 2917 | 1119 | 0.384 |
| Extract | | | |
| Concentrated | 17133 | 6574 | 0.384 |
| Extract | | | |
| 6 × Diafiltered | 25166 | 3767 | 0.150 |
| against AMBI | | | |
| 10 × Diafiltered | 13967 | 1135 | 0.081 |
| against AMBI | | | |
| 20 × Diafiltered | 17965 | 281 | 0.016 |
| against AMBI | | | |
| ˜5 × Diafiltered | 4648 | 1596 | 0.343 |
| against Water | | | |

The use of water as the diafiltration liquid is contraindicated due to the fouling of the filter and the retention of impurities removed using 100 mM ammonium bicarbonate.

Scale-up of the PI2 isolation process

A method to isolate and purify PI2 from potatoes on a commercial scale is described. Pilot trials conducted to examine the process steps and sampling points as outlined above demonstrated the effectiveness and reproducibility of the method of the present invention. During pilot trials, intermediates were generated and tested by quality control methods as described herein. Pilot experiments were designed and implemented with an eye toward scalability to full commercial production. Data from process optimization and production capability verification is summarized below.

1. Raw Material Choice

Whole, raw potatoes were used for the protease inhibitor feedstock. The Russet Norkotah and Norkotah varieties consistently exhibited levels of PI2 below the 100 mg/kg target. The Russet Burbank materials examined showed large variances variability in PI2 content. To have a consistent feedstock source, a survey of Russet Burbank potatoes was completed. While numerous suppliers offered materials that attained the 100 mg/kg target (Green Giant, Kingston, Idaho Gem), one supplier (Green Giant, Potandon Produce, Idaho Falls, Id.) consistently delivered materials that exceeded the target yield. In addition to varietal and supplier considerations, pilot runs were performed using different sized potatoes (“count”). The optimum yields were generated using 90 count potatoes.

2. Formic acid content

Many organic acids were examined for extraction efficiency and an acid that consistently exhibited the greatest cost-effectiveness. Formic acid lowers the pH of the potato slurry generated in the grinding of the feedstock potatoes. Lowering slurry pH increases the solubility of PI2, while reducing microbiological growth and browning of materials during processing. Extractant solution formulations having formic acid content from 0.5% to 2.5% have been examined and optimal results were obtained using a solution containing 1.5% formic acid. While a decline in PI2 yield was not observed under extractant solution conditions utilizing greater than 1.5% formic acid content, higher acid concentrations achieved no discernable benefit at solution levels higher than 1.5%.

3. Sodium chloride content

Sodium chloride concentrations from zero to 2.0 N were examined for PI2 extraction efficiency and protein purification capability. A 1.0 N salt concentration was determined to be optimal and sodium chloride concentrations below 0.3 N exhibited a reduced capability to remove impurities during the extraction process. PI2 extraction efficiency was maximized using extract solutions of 1.0 N or greater.

4. Extractant to raw potato ratio

Extractant to raw potato weight ratios from 1:1 to 1:10 were studied. The minimum amount of extractant necessary for efficient extraction of PI2 was observed at 1:2.5 w/w (one part extractant to two and one-half parts raw potato, by weight).

5. Grind profile of the final slurry

The potato and extractant solvent slurry is comminuted in a grinder. Increased grind time generated a grind profile in which the resulting average particle size was approximately 500 μm. A 500 μm particle size allowed for greater contact between the raw potato solids and therefore resulted in enhanced PI2 yields. Minimum grind profile is limited and established by two factors. First, a finer grind generates increased heat in the grinding head and while PI2 is heat stable, losses can occur during extended periods of elevated temperature. Second, a larger proportion of finely ground particles in the slurry adversely affected subsequent separation steps.

6. Screen mesh of the bulk centrifuge

A 35 μm bag mesh demonstrated optimal qualities. An approximate seventy-five percent load allows for rapid separation, without blinding of the mesh screen by potato starch particles.

7. Maximum temperature of the material during the heat treatment

The target protein (PI2) is stable at elevated temperatures, in comparison to other proteins commonly found in potato varieties. PI2 is reported in the literature as heat stable for up to three hours at 70° C. At 70° C., other impurities in the filtrate begin to exhibit decreased solubility, whereas PI2 begins to exhibit signs of denaturation at extended exposure to temperatures of around 80° C. To minimize the heat-related degradation of PI2, maximum filtrate temperature is capped at 70° C.

8. Hold time of the heat treatment

While non-heat stable impurities began to alter in solubility at 70° C., the amount of impurity altered increased directly with the time the filtrate remained at 70° C. No increase in impurity precipitation was observed beyond 60 minutes, and the PI2 exhibited no yield loss over a 60-minute treatment at 70° C.

9. Final temperature of the cooling stage

No significant increase in the amount of impurity precipitate is achieved by cooling below 25° C. The PI2 is fully soluble in the temperature range of 20° to 25° C., so no decrease in recovery is associated with filtrate cooling.

10. Hold-time/force applied in centrifuge clarification

Exposing the solution to roughly 13,000 times gravity (×g) for approximately 90 seconds achieved separation, removing approximately two percent of the heat treated filtrate as impurities. Heat-treated impurities removed via centrifugation at 13,000 ×g were devoid of PI2.

11. Percent solids allowed in clarified extract

While the clarifier can operate with a feed rate from 0.1 liter per minute to 2.0 liters per minute, an optimal flow rate of 1.5 liters per minute generated a clarified extract containing less than 0.01% solids by weight, a particulate level that did not adversely affect extract microfiltration.

12. Pore size of microfilter

A solution filtered to extract particles 0.2 μm or larger will help reduce the potential for micro-bacteriological contamination. Attempts to microfilter the solution below 0.3 μm were unsuccessful. With a 0.01% solids by weight particulate level from the clarifier, microfiltration with a 0.3 μm filter was considered ideal.

13. Membrane composition

A cellulosic, open, screen-channel membrane exhibited tolerance for the pH and ionic strength of the clarified and filtered extract. Pilot trials demonstrated that a cellulosic, open, screen-channel membrane will continue to function properly so long as the membrane is kept clean.

14. Molecular weight cut-off

A molecular weight cut-off rated for 10 KD was determined to minimize the loss of PI2 through the membrane, while removing a large percentage of the impurities. A membrane rated greater than 10KD allowed PI2 to pass through the membrane, whereas a 5KD membrane did not remove the majority of the impurities present in the extract.

15. Concentration factor

The maximum practical concentration was observed where one-tenth of the starting volume remained as the concentrated extract, a point at which the concentrated extract precipitate begins to form on the membrane surface, eventually leading to membrane impermeability.

16. Flow rate

The flow rate was optimized to 0.40 liters per minute per square foot of membrane surface, with a twenty psi pressure differential. Lower flow rates did not generate sufficient pressure to maintain maximum permeate collection. Higher flow rates resulted in the deposition of high molecular weight impurities in the screen-channel openings, thereby fouling the filter and dramatically slowing the entire ultrafiltration process.

17. Choice of buffer for diafiltration

Ammonium bicarbonate was selected as an ideal diafiltration buffer based on the following criteria: pH, salt exchange ability, and ease of removal. Diafiltration against an ammonium bicarbonate buffer establishes a final pH near neutral (pH ˜7.8). In the extract, ammonium bicarbonate binds with PI2 allowing for further sodium chloride removal from the ultrafiltered extract. Most importantly, ammonium bicarbonate is completely removed during the final drying of the ultrafiltered solution. This results in a near-native form of PI2 at the end of the isolation scheme.

18. Buffer concentration

Use of ammonium bicarbonate was minimized due to cost and disposal considerations. The lowest buffer concentration effective in pH change and product purity was 100 mM.

19. Volume of buffer used in diafiltration

Fewer washes generated an ultrafiltered product that exhibited higher impurity content. Six volumes of 100 mM ammonium bicarbonate were determined to maximize the purification utility of the diafiltration. Nominal removal of impurities was observed beyond six washes.

Conclusions

Ultrafiltration optimization required choosing the correct membrane material. The filter material choices were either cellulose or polyethersulfone. It was found that either of these materials was acceptable, with the cellulose being the better choice due to decreased blinding during ultrafiltration and equal ease of cleaning after usage. The molecular weight cutoff for filter selection is either 5,000 Daltons or 10,000 Daltons. Either of these pore sizes was suitable for retaining the PI2, which has a molecular mass of 21,000 Daltons. It was found that no PI2 was lost through the 10,000 Dalton filter and that ultrafiltration rates were approximately twice as fast in comparison to the 5,000 Dalton filter. Total cross flow rates of greater than 7 liters/(meter²-hour) of filter with a back pressure of 20-25 psig afforded the fastest ultrafiltration with no membrane fouling. Cleaning the membrane with a solution of 0.1 N sodium hydroxide, as recommended by the manufacturer, was sufficient to maintain an optimal filtration rate. The use of water as a diafiltration buffer was contraindicated due to rapid fouling of the filter. The use of 100 mM ammonium bicarbonate as a diafiltration buffer prevented fouling by presenting an ionic strength sufficient to discourage precipitation of solids onto the filter. Ammonium bicarbonate also promoted the removal of some impurities, and was readily removed by drying, e.g., lyophilization.

The results of the studies described herein demonstrate that for reproducibility, maximum yield, impurity removal, minimal cost, and maximum commercial feasibility, the conditions described herein are ideally suited for achieving a desired result. An ideal membrane composition comprises regenerated cellulose utilizing Maximate geometry with a nominal molecular weight cut-off of 10,000 Dalton. A flow rate across the regenerated cellulose membrane of 60 LMH at 0.4 L/minute per membrane square foot is ideal to achieve a concentration factor of 10 times volume of the clarified extract volume when using an ammonium bicarbonate diafiltration buffer at a working concentration of 100 mM diafiltered against six times the concentrated volume.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto; except insofar as the claims are so limited. Those skilled in the art who have the disclosure before them will be able to make modifications and variations therein without departing from the scope of the invention.

What is claimed is:

1. A method for isolation and purification of a proteinase inhibitor from tissue of a plant, comprising the steps of:
(a) extracting the protease inhibitor and other protein products from the plant tissue by preparing a mixture of solvent and comminuted plant tissue to form a solid fraction and a liquid fraction comprising the protease inhibitor and other protein products;
(b) separating the liquid fraction from the solid fraction;
(c) heating the liquid fraction to a temperature and for a time period sufficient to denature at least some of the other protein products without denaturing the protease inhibitor;
(d) cooling the liquid fraction to reduce the solubility of the denatured protein products; and
(e) removing the denatured protein products to prepare a clarified extract solution;
(f) filtering the extract solution in the presence of a buffer to form a retentate solution having a pH of less than about 8.0 and to remove non-denatured protein products.

2. The method of claim 1 wherein the microfiltration filter pore size is between about 0.2 μm and about 0.5 μm.

3. The method of claim 1 wherein ultrafiltration is conducted on an open, screen-channel membrane having a molecular weight cut-off rating of about 5 KD to about 10 KD.

4. The method of claim 10 wherein the ultrafiltration buffer has a conductivity of less than 7.5 mS/mol.

5. The method of claim 1 wherein the filtration buffer comprises an aqueous solution of ammonium bicarbonate.

6. The method of claim 5 wherein the filtration buffer is between about 50 and about 500 mM ammonium bicarbonate.

7. The method of claim 1 wherein the retentate solution is concentrated to less than one-fifth of the starting volume during filtration.

8. The method of claim 7 wherein the filtration comprises ultrafiltration using a membrane and is conducted at a rate of between about 0.20 liters and about 0.6 liters per minute per square foot of membrane surface.

9. The method of claim 8 wherein the filtration step further comprises washing with up to ten volumes of filtration buffer.

10. The method of claim 8 wherein the membrane is selected from a group consisting of regenerated cellulose and polyethersulfone.

11. A method for isolation and purification of a proteinase inhibitor from tissue of a plant containing the protease inhibitor and other protein products, comprising the steps of:
(a) extracting the protease inhibitor and other protein products from the plant tissue by preparing a mixture of solvent and the plant tissue and comminuting to a particulate size of between about 100 μm to about 1000 μm to form a solid fraction and a liquid fraction comprising the protease inhibitor and other protein products;
(b) separating the liquid fraction from the solid fraction;
(c) heating the liquid fraction to a temperature and for a time period sufficient to denature at least some of the other protein products without denaturing the protease inhibitor;
(d) cooling the liquid fraction to reduce the solubility of the denatured protein products;
(e) removing the denatured protein products to prepare a clarified extract solution; and
(f) filtering the extract solution in the presence of a buffer of less than about 10 mS/mol conductivity to form a retentate solution and to remove non-denatured protein products.

12. A method for isolation and purification of a proteinase inhibitor from potato tubers containing the protease inhibitor and other protein products, comprising the steps of:
(a) preparing an aqueous extractant solution of formic acid and sodium chloride, adding to the extractant solution a quantity of the potato tubers, and mixing to form a slurry using a grind profile to result in a predetermined average particle size of the potato in the extractant slurry;
(b) centrifuging the slurry to form a filtrate containing the protease inhibitor and the other protein products;
(c) denaturing the other protein products by heat treating the filtrate;
(d) precipitating the denatured proteins by cooling the filtrate and separating them by centrifugation to form a clarified extract;
(e) filtering the clarified extract through a microfilter and collecting the resulting extract solution; and
(f) filtering by ultrafiltration the extract solution with an ultrafiltration buffer to form a retentate solution.

13. The method of claim 12 wherein the microfiltration filter pore size is between about 0.15 μm and about 0.4 μm.

14. The method of claim 12 wherein ultrafiltration is conducted on an open, screen-channel membrane having a molecular weight cut-off rating of about 5 KD to about 10 KD.

15. The method of claim 14 wherein the ultrafiltration buffer has a conductivity of less than 5.5 mS/mol.

16. The method of claim 14 wherein the ultrafiltration buffer comprises an aqueous solution of ammonium bicarbonate.

17. The method of claim 16 wherein the ultrafiltration buffer is 100 mM ammonium bicarbonate.
